(19) United States
(12) Patent Application Publication
(10) Pub. No.: US 2024/0374870 A1
(43) Pub. Date: Nov. 14, 2024

(54) GUIDEWIRE AND METHOD OF USE

(71) Applicant: Abbott Cardiovascular Systems Inc., Santa Clara, CA (US)

(72) Inventors: Puneet Kamal Singh Gill, Anaheim, CA (US); Robert Hayzelden, Murrieta, CA (US); Stephanie Maldonado, Winchester, CA (US); Jessica Marie Saenz, Lake Elsinore, CA (US); Matthew Vasquez, Temecula, CA (US)

(73) Assignee: Abbott Cardiovascular Systems Inc., Santa Clara, CA (US)

(21) Appl. No.: 18/144,607

(22) Filed: May 8, 2023

(51) Int. Cl. A61M 25/09

(52) U.S. Cl. CPC A61M 25/09; A61M 2025/09133; A61M 2025/09175

(57) ABSTRACT

A guidewire for use in the vasculature is formed inside a vessel into an extended U-shaped section at the distal end. The extended U-shaped distal section is able to more easily advance through tortuous vasculature or calcified lesions.

### BACKGROUND

The technology herein relates to the field of guidewires for advancing intraluminal devices such as stent delivery catheters, balloon dilatation catheters, atherectomy catheters and the like within body lumens.

In a typical coronary procedure a guiding catheter having a preformed distal tip is percutaneously introduced into a patient's artery, e.g., femoral or brachial artery, by means of a conventional Seldinger technique and advanced therein until the distal tip of the guiding catheter is seated in the ostium of a desired coronary artery. There are two basic techniques for advancing a guidewire into the desired location within the patient's coronary anatomy, the first is a preload technique which is used primarily for over-the-wire (OTW) devices and the second is a bare wire technique which is used primarily for rapid exchange type systems. With the preload technique, a guidewire is positioned within an inner lumen of an OTW device such as a dilatation catheter or stent delivery catheter with the distal tip of the guidewire just proximal to the distal tip of the catheter and then both are advanced through the guiding catheter to the distal end thereof. The guidewire is first advanced out of the distal end of the guiding catheter into the patient's coronary vasculature until the distal end of the guidewire crosses the arterial location where the interventional procedure is to be performed, e.g., a lesion to be dilated or a dilated region where a stent is to be deployed. The catheter, which is slidably mounted onto the guidewire, is advanced out of the guiding catheter into the patient's coronary anatomy over the previously introduced guidewire until the operative portion of the intravascular device, e.g., the balloon of a dilatation or a stent delivery catheter, is properly positioned across the arterial location. Once the catheter is in position with the operative means located within the desired arterial location, the interventional procedure is performed. The catheter can then be removed from the patient over the guidewire. Usually, the guidewire is left in place for a period of time after the procedure is completed to ensure reaccess to the arterial location.

With the bare wire technique, the guidewire is first advanced by itself through the guiding catheter until the distal tip of the guidewire extends beyond the arterial location where the procedure is to be performed. Then a rapid exchange (RX) catheter is mounted onto the proximal portion of the guidewire which extends out of the proximal end of the guiding catheter, which is outside of the patient. The catheter is advanced over the guidewire, while the position of the guidewire is fixed, until the operative means on the RX catheter is disposed within the arterial location where the procedure is to be performed. After the procedure, the intravascular device may be withdrawn from the patient over the guidewire or the guidewire advanced further within the coronary anatomy for an additional procedure.

Conventional guidewires for angioplasty, stent delivery, atherectomy and other vascular procedures usually comprise an elongated core member with one or more tapered sections near the distal end thereof and a flexible body such as a helical coil or a tubular body of polymeric material disposed about the distal portion of the core member. A shapeable member, which may be the distal extremity of the core member or a separate shaping ribbon, which is secured to the distal extremity of the core member, extends through the flexible body and is secured to the distal end of the flexible body by soldering, brazing or welding which forms a rounded distal tip. Torqueing means are provided on the proximal end of the core member to rotate, and thereby steer, the guidewire while it is being advanced through a patient's vascular system.

It is typical that best medical practice for anatomical insertion requires a guidewire that has behavioral characteristics that vary along its length. For example, under some conditions, the distal end of the guidewire may be required to be more flexible than the proximal end so that the distal end may more easily be threaded around the more tortuous distal branches of the luminal anatomy. Further, the proximal end of the guidewire may be required to have greater torsional stiffness than the distal end because, upon rotation of the guidewire, the proximal end must carry all the torsional forces that are transmitted down the length of the guidewire, including what is required to overcome cumulative frictional losses.

For certain procedures, such as when delivering stents around a challenging take-off, e.g., a shepherd's crook, tortuosities or severe angulation, substantially more support and/or vessel straightening is frequently needed from the guidewire than normal guidewires can provide. Guidewires have been commercially introduced for such procedures which provide improved distal support over conventional guidewires, but such guidewires are not very steerable and in some instances are so stiff that they can damage vessel linings when advanced therethrough. What has been needed and heretofore unavailable is a guidewire which provides a high level of distal support with acceptable steerability and little risk of damage when advanced through a patient's vasculature.

### SUMMARY OF THE INVENTION

Guidewires are designed based on specific applications and usage. Most guidewires are designed to track while traversing the vasculature, and not prolapse within the vessel or into side branches. In practice, however, physicians may desire guidewires that are intentionally designed to prolapse (i.e., knuckle) while tracking for specific clinical scenarios, which can be achieved by design modifications to the distal section of the guidewire. Importantly, when designing a guidewire that will form a knuckle or U-shaped section on the distal end of the guidewire, the knuckle must form a tight radius (U-shape) that will only grow to a certain length.

In one embodiment, a guidewire is formed of an elongated tabular member having a proximal end and a distal end. A proximal section extends from the proximal end toward the distal end and ends where the elongated tubular member begins to taper toward the distal end. A distal section extends from the point where the taper begins and ends at the distal end of the guidewire. The distal section includes one or more tapered sections and a constant diameter section. A transition joint is positioned on the constant diameter section between the tapered section and the distal end of the guidewire. An extended U-shaped section is formed adjacent the transition joint and has an outer curve having an arc length and a midpoint on the arc length. The extended U-shaped section has a first member having a first length, a second member having a second length, and a U-shaped bend joining the first member and the second member. The first member extends from the distal end of the elongated tubular member to the midpoint on the arc length, and the second length of the second member is equal to the first length of the first member. The second length of the second member extends from the midpoint of the arc length and along the constant diameter section of the distal section. In one embodiment, the constant diameter section has a weakened section on the second member and adjacent the transition joint. In another embodiment, the constant diameter section has a strengthened section on the second member and adjacent the transition joint.

In another embodiment, a guidewire has all of the structure recited above, except the constant diameter section is a constant rectangular section.

As disclosed further herein, there are multiple embodiments for forming the weakened and strengthened sections. Importunately, in all of the embodiments, after the U-shaped section is formed, the first member and the second will not grow in length.

### BRIEF DESCRIPTION OF THE DRAWINGS

FIG. 1 is an elevational view of a prior art guidewire.

FIG. 2 is an elevational view, partially in section, of a prior art guidewire.

FIG. 3 is an elevational view of the U-shaped guidewire of the invention navigating a tortuous vessel.

FIG. 4A is an elevational view of a guidewire having a weakened transition point at the transition between a tapered section and a constant diameter section.

FIG. 4B is a cross-sectional view taken along lines 4B-4B, depicting a round cross-section on the constant diameter section.

FIG. 4C is a cross-sectional view taken along lines 4C-4C depicting a rectangular cross-section on the constant rectangular section.

FIG. 4D is an elevational view of a guidewire having a weakened transition point at a heat treated area between a tapered section and a constant diameter section.

FIG. 4E is an elevational view of a guidewire having a strengthened transition point at a heat treated area between a tapered section and a constant diameter section.

FIG. 4F is an elevational view of the guidewire of FIGS. 4A-4C depicting a U-shaped section formed at the transition point.

FIG. 4G is a graph depicting standard heat treatment data reported in The Effects of Cold Work and Heat Treatment on the Properties of Nitinol Wire, by M. Drexel, G. Selvaduray and A. Pelton: BioMed 2007.

FIG. 5A is an elevational view of a guidewire depicting a strengthened transition point.

FIG. 5B is an elevational view of the guidewire of FIG. 5A depicting a U-shaped section formed at the strengthened transition points wherein the U-shaped section does not lengthen beyond the strengthened transition points.

FIG. 6A is an elevational view of a guidewire depicting a straight flat ribbon or wire having a strengthened transition point.

FIG. 6B is an elevational view of the guidewire of FIG. 5A depicting a U-shaped section formed at the strengthened transition points.

FIG. 6C is a cross-sectional view taken along lines 6C-6C depicting the rectangular shape of the hybrid ribbon wire.

FIG. 6D is a cross-sectional view taken along lines 6D-6D depicting the rectangular shape of the hybrid ribbon wire and the distal section of the guidewire.

FIG. 7A is an elevational view of a guidewire having a weakened transition point in the form of a notch.

FIG. 7B is an elevational view of the guidewire of FIG. 7A depicting a U-shaped section formed at the notch.

FIG. 8A is an elevational view of a guidewire depicting a weakened transition point in the form of a bellows joint.

FIG. 8B is an elevational view of a guidewire depicting a weakened transition point in the form of a bellows joint.

FIG. 8C is an elevational view of a guidewire depicting a weakened transition point in the form of a bellows joint.

FIG. 8D is an elevational view of the guidewire shown in FIGS. 8A and 8B depicting a U-shaped section formed at the bellows joint.

### DETAILED DESCRIPTION OF THE PREFERRED EMBODIMENTS

### Prior Art Guidewires

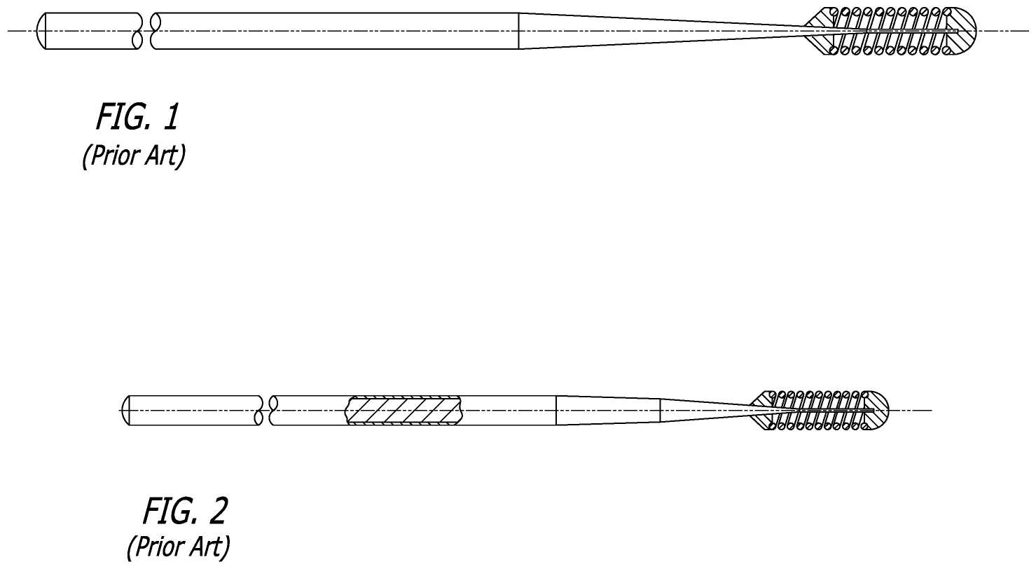

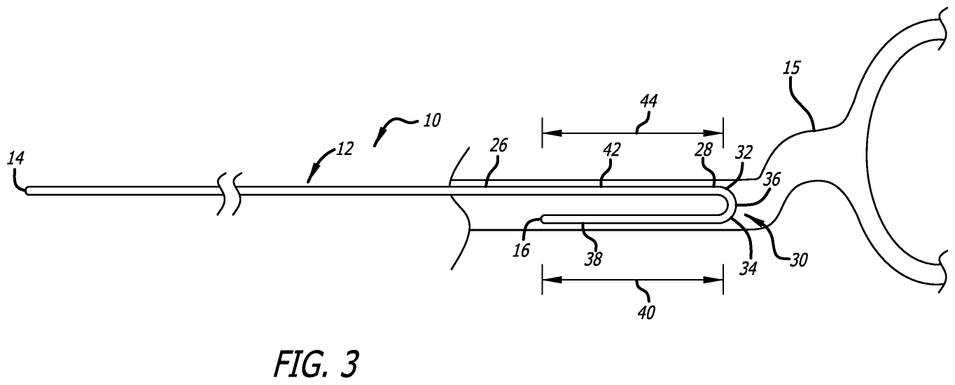

Prior art guidewires typically include an elongated core wire having a flexible atraumatic distal end. A prior art guidewire is shown in FIGS. 1 and 2 and includes an elongated core member with a proximal core section, a distal core section, and a flexible body member which is fixed to the distal core section. The distal core section has a tapered segment, a flexible segment which is distally contiguous to the tapered segment, a distal end, and a proximal end. The distal section may also have more than one tapered segment which have typical distally decreasing tapers with substantially round transverse cross sections.

The core member may be formed of stainless steel, NiTi alloys or combinations thereof. The core member is optionally coated with a lubricious coating such as a fluoropolymer, e.g., TEFLON®, which extends the length of the proximal core section. Hydrophilic coatings may also be employed. The length and diameter of prior art guidewire may be varied to suit the particular procedures in which it is to be used and the materials from which it is constructed. The length of the guidewire generally ranges from about 65 cm to about 320 cm, more typically ranging from about 160 cm to about 200 cm, and preferably from about 175 cm to about 190 cm for the coronary anatomy. The guidewire diameter generally ranges from about 0.008 inch to about 0.035 inch (0.203 to 0.889 mm), more typically ranging from about 0.012 inch to about 0.018 inch (0.305 to 0.547 mm), and preferably about 0.014 inch (0.336 mm) for coronary anatomy.

The flexible segment terminates in a distal end. Flexible body member, preferably a coil, surrounds a portion of the distal section of the elongated core, with a distal end of the flexible body member secured to the distal end of the flexible segment by the body of solder. The proximal end of the flexible body member is similarly bonded or secured to the distal core section by a body of solder. Materials and structures other than solder may be used to join the flexible body to the distal core section, and the term “solder body” includes other materials such as braze, epoxy, polymer adhesives, including cyanoacrylates, weld, glue and the like.

In FIG. 2, the prior art guidewire has a core member, a helical coil, a first tapered core segment, and a second tapered core segment, which is distally contiguous to the first tapered core segment. The second tapered core segment tapers at a greater degree than the first tapered core segment and this additional taper provides a much smoother transition when the distal portion of the guidewire is advanced through a tortuous passageway. The degree of taper of the first tapered core segment, i.e., the angle between the longitudinal axis and a line tangent to the first tapered core segment typically is about 0.146°, whereas the taper of the second tapered core segment, i.e., the angle between the longitudinal axis and the second tapered core segment is larger than the first angle and typically is about 0.109° such as is shown in the view of the guidewire in FIG. 2. Moreover, all of the multiple tapered core segments need not have increasing degrees of tapers in the distal direction. However, two or more contiguous tapered core segments over a length of about 5 to 15 cm should have distally increasing degrees of tapering.

The core member is coated with a lubricious coating such as a fluoropolymer, e.g., TEFLON®, which extends the length of the proximal segment. The distal portion is also provided a lubricous coating, not shown for purposes of clarity, such as a MICROGLIDE™ coating used by the present assignee, Abbott Cardiovascular Systems, Inc., on many of its commercially available guidewires. A hydrophilic coating may also be employed.

The core member may be formed of stainless steel, CoCr, Ti, and NiTi alloys or combinations thereof or other high strength alloys as is well known in the art.

### U-Shaped Guidewire Design

Guidewires are designed based on specific applications and usage. Most guidewires are designed to track while traversing the vasculature, and not prolapse within the vessel or into side branches. In practice, however, physicians may desire guidewires that are intentionally designed to prolapse (i.e., knuckle) while tracking for specific clinical scenarios, which can be achieved by design modifications to the distal section of the guidewire. Importantly, when designing a guidewire that will form a knuckle or U-shaped section on the distal end of the guidewire, the knuckle must form a tight radius (U-shape) that will only grow to a certain length.

The guidewire of the present invention is designed to track the vasculature and form a knuckle (U-shaped section) in a vessel due to calcification, resistance, fibrous tissue, etc. The knuckle forms in the vessel when a pressure or force is applied at the distal tip of the guidewire.

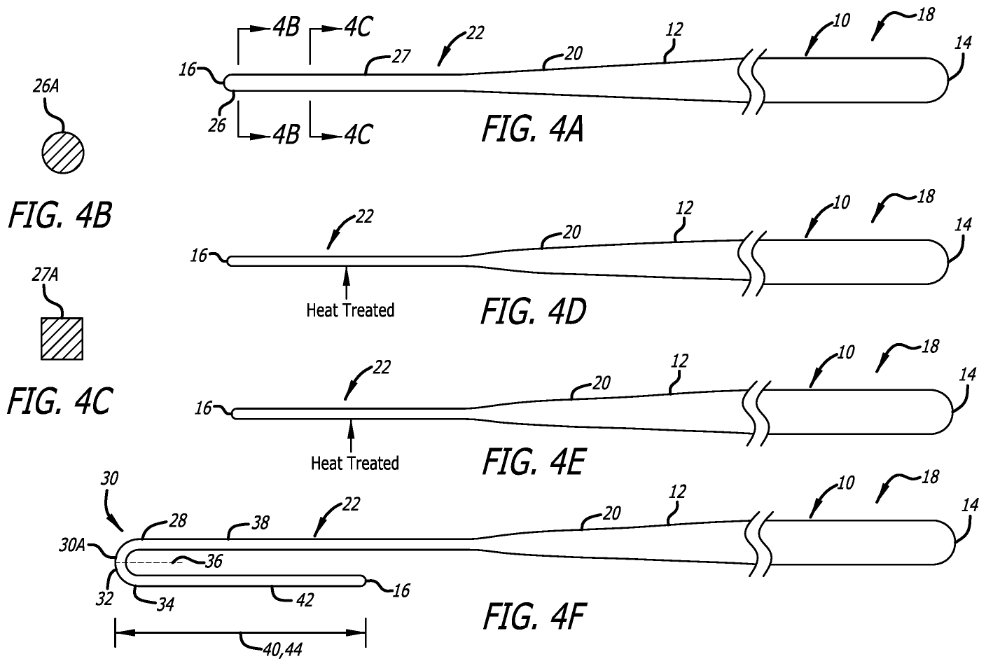

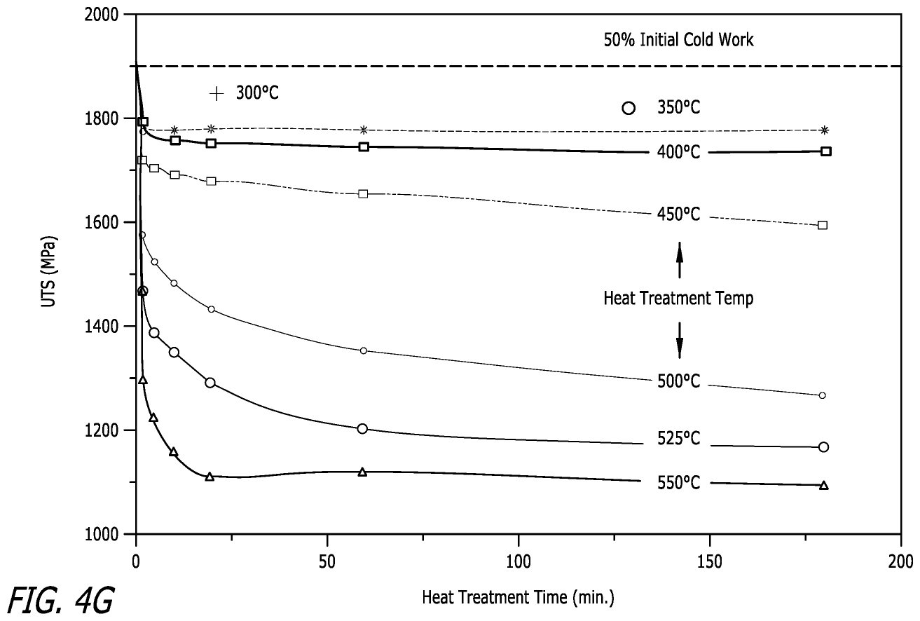

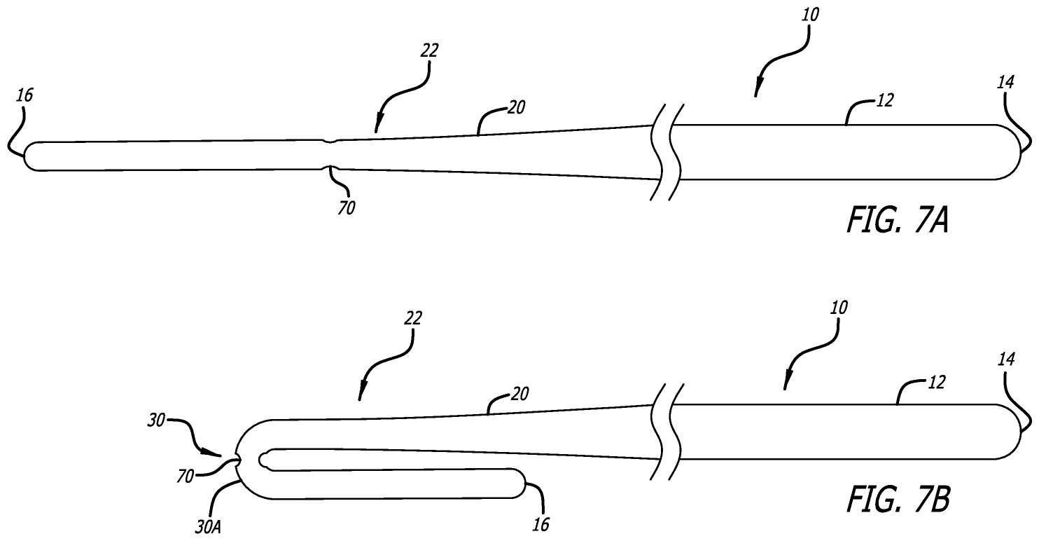

In one embodiment, as shown in FIGS. 3, 4A, 4B and 5A-8D, a guidewire 10 is formed of an elongated tubular member 12 having a proximal end 14 and a distal end 16 for use in navigating tortuous vessels 15. A proximal section 18 extends from the proximal end 14 toward the distal end 16 and ends where the elongated tubular member 12 begins a tapered section 20 toward the distal end 16. More specifically, the proximal section 18 extends from the proximal end 14 of the elongated tubular member 12 to a proximal end 21A of the tapered section 20. A distal section 22 extends from the point where the tapered section 20 begins, and ends at the distal end 16 of the guidewire 10. More specifically, the distal section 22 extends from the proximal end 21A of the tapered section 20, toward a distal end 21B (sec FIGS. 6A and 6B) of the tapered section 20, and ends at the distal end 16 of the elongated tubular member 12. The distal section 22 includes one or more tapered sections 20 and a constant diameter section 26. In this embodiment, the constant diameter section 26 has a round cross-section 26A, as shown in FIGS. 4A and 4B. A transition joint 28 is positioned on the constant diameter section 26 between the tapered section 20 and the distal end 16 of the guidewire 10. An extended U-shaped section 30 is formed adjacent the transition joint 28 and has an outer curve 32 having an arc length 34 and a midpoint 36 on the arc length 34. The extended U-shaped section 30 has a first member 38 having a first length 40, a second member 42 having a second length 44, and a U-shaped bend 46 joining the first member 38 and the second member 42. The first member 38 extends from the distal end 16 of the elongated tubular member 12 to the midpoint 36 on the arc length 34, and the second length 44 of the second member 42 is equal to the first length 40 of the first member 38. The second length 40 of the second member 42 extends from the midpoint 36 of the arc length 34 and along the constant diameter section 26 of the distal section 22. In one embodiment, the constant diameter section 26 has a weakened section 50 on the second member 42 and adjacent the transition joint 28. There are multiple embodiments of the weakened section 50 as further disclosed herein.

In another embodiment, as shown in FIGS. 3, 4A, 4C and 5A-8D, a guidewire 10 is formed of an elongated tubular member 12 having a proximal end 14 and a distal end 16. A proximal section 18 extends from the proximal end 14 toward the distal end 16 and ends where the elongated tubular member 12 begins a tapered section 20 toward the distal end 16. More specifically, the proximal section 18 extends from the proximal end 14 of the elongated tubular member 12 to a proximal end 21A of the tapered section 20. A distal section 22 extends from the point where the tapered section 20 begins and ends at the distal end 16 of the guidewire 10. More specifically, the distal section 22 extends from the proximal end 21A of the tapered section 20, toward a distal end 21B of the tapered section 20, and ends at the distal end of the elongated tubular member 12. The distal section 22 includes one or more tapered sections 20 and a constant rectangular section 27. In this embodiment, the constant rectangular section 27 has a rectangular cross-section 27A as shown in FIGS. 4A and 4C. A transition joint 28 is positioned on the constant rectangular section 27 between the tapered section 20 and the distal end 16 of the guidewire 10. An extended U-shaped section 30 is formed adjacent the transition joint 28 and has an outer curve 32 having an arc length 34 and a midpoint 36 on the arc length 34. The extended U-shaped section 30 has a first member 38 having a first length 40, a second member 42 having a second length 44, and a U-shaped bend 46 joining the first member 38 and the second member 42. The first member 38 extends from the distal end 16 of the elongated tubular member 12 to the midpoint 36 on the arc length 34, and the second length 44 of the second member 42 is equal to the first length 40 of the first member 38. The second length 44 of the second member 42 extends from the midpoint 36 of the arc length 34 and along the constant rectangular section 27 of the distal section 22. In one embodiment, the constant rectangular section 27 has a weakened section 50 on the second member 42 and adjacent the transition joint 28. The constant rectangular section 27 can be formed by grinding a round diameter wire so that a first flat has a 90° orientation to a second flat. In one embodiment, the constant rectangular section 27 can be formed using flattening dies in a known manner. There are multiple embodiments of the weakened section 50 as further disclosed herein.

In one embodiment, which includes either the constant diameter section 26 (FIG. 4B) or the constant rectangular section 27 (FIG. 4C), the weakened section 50 (FIG. 4D) is formed by heat treating a portion of the distal section 22. More specifically, localized heat of 500° C. is applied to the area of the transition joint 28 for 60 minutes, which will make the material softer due to annealing (i.e., dominate the precipitation strengthening effect and causing a drop in ultimate tensile strength). Since the annealed section at the transition joint 28 is softer, this forms the weakened section 50 so that the guidewire can be easily bent to form the U-shaped section 30 (see FIG. 4E). Depending on the material used to make the guidewire 10, the heat and time applied to the wire can range from 350° C. to 600° C. and last from 30 seconds to 85 minutes.

In another embodiment, which includes either the constant diameter section 26 (FIG. 4B) or the constant rectangular section 27 (FIG. 4C), a strengthened section 54 (FIG. 4E) is formed by treating a portion of the distal section 22. More specifically, localized heat of 350° C. is applied to the area adjacent the transition joint 26 for 30 minutes, which will make the material stiffer (i.e., precipitates acting as barriers to dislocation motion). The heat treatment forms the strengthened section 54 so that portion of the guidewire 10 that is distal of the strengthened section 54 is relatively weaker and can be bent at the transition joint 28 to form the U-shaped section 30 (see FIG. 4F). All of these embodiments promote formation of the knuckle or U-shaped section 30.

Depending on the material used to form the guidewire 10, the heat and the time applied to the wire can range from 250° C. to 450° C. and last for 2 minutes to 45 minutes. The heat can be applied to one or more sections of the guidewire 10 in order to facilitate forming the U-shaped section 30. Based on the desired functionality, a single section or multiple sections of the distal section 22 of the guidewire 10 can be exposed to the disclosed heat treatments. The guidewire 10 can be heat treated by various means including laser treatment to alter the metallic properties.

The graph shown in FIG. 4G illustrates the effect of temperature and time on the ultimate tensile strength (UTS). As an example, the higher the temperature (500° C.) and the longer the time (60 minutes) that the heat is applied, the ductile/less stiff the wire gets (lower UTS).

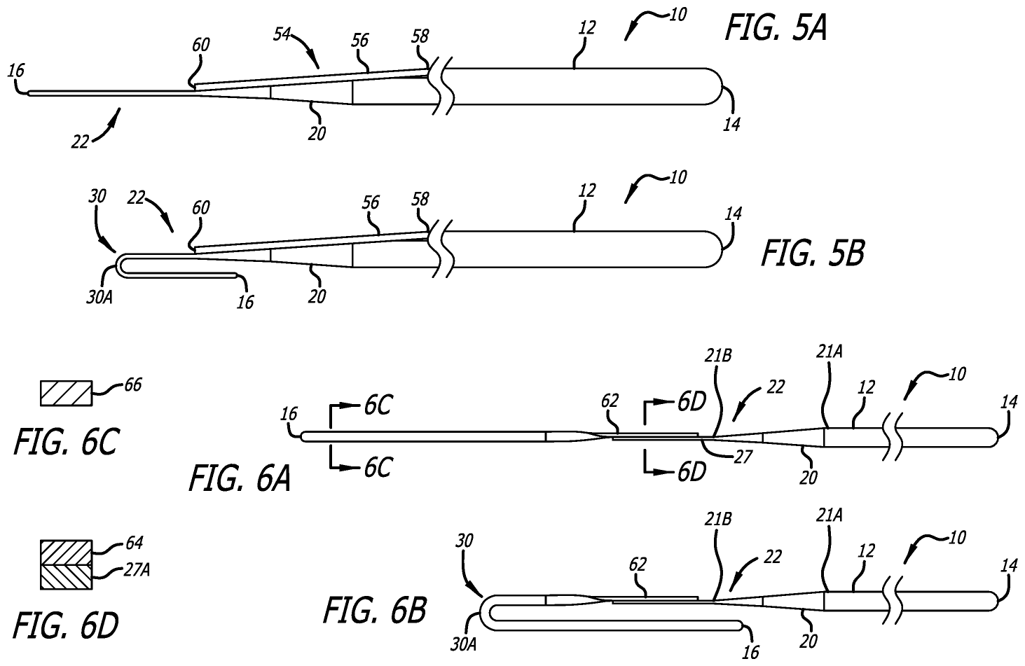

In another embodiment, shown in FIGS. 5A and 5B, the guidewire 10 has a strengthened section 54 formed by attaching a ribbon wire (or ribbon coil) 56 to the distal section 22. The ribbon wire (or ribbon coil) 56 can be attached to the distal section 22 by various means including soldering, welding, brazing, spot weld with a laser, and adhesives. If a ribbon coil 56 is used, it can slide over the distal section 22 and it will stay in place without further attachment means. A short section of hollow hypotube (not shown) can be used to receive the ribbon wire 56 and the distal end 16 of the distal section 22 in order to attach the ribbon wire 56 to the distal section 22. The ribbon wire 56 has a proximal end 58 and a distal end 60. The distal section 22 is strengthened by the attachment of the ribbon wire 56 so that the portion of the distal section 22 that is distal of the distal end 60 of the ribbon wire 56 is weaker and easily bendable. Thus, the transition joint 26 is just distal of the distal end 60 of the ribbon wire 22 and the U-shaped section 30 is formed by bending the wire at the transition joint 28. The ribbon wire 56 can be attached to either the round cross-section 26A of the constant diameter section 26 or the rectangular cross-section 27A of the constant rectangular section 27. In one embodiment, the ribbon wire 56 has a rectangular cross-section and it is attached in the opposite plane of the flattened rectangular cross-section 27A of the constant rectangular section 22. In one embodiment, the ribbon wire 56 can have a 90° twist where the twist falls at the attachment point (i.e., the 90° twist falls at the weld, solder, brazed area).

In another embodiment, as shown in FIGS. 6A and 6B, a hybrid ribbon wire 62 is used to form the strengthened section 54 in guidewire 10. The hybrid ribbon wire 62 has a rectangular cross-section proximal section 64 that is attached to a rectangular cross-section distal section 66 by any of welding, soldering, brazing, spot welding with a laser, and adhesives. A short section of hollow hypotube (not shown) can be used to receive the ribbon wire 56 and the distal end 16 of the distal section 22 in order to attach the ribbon wire 56 to the distal section 22. The hybrid ribbon wire 62 has a rectangular cross-section distal section 66 that extends distally of the distal end 16 of the distal section 22 of the guidewire 10. The rectangular cross-section distal section 66 can be bent at the transition joint 28 to form the U-shaped section 30.

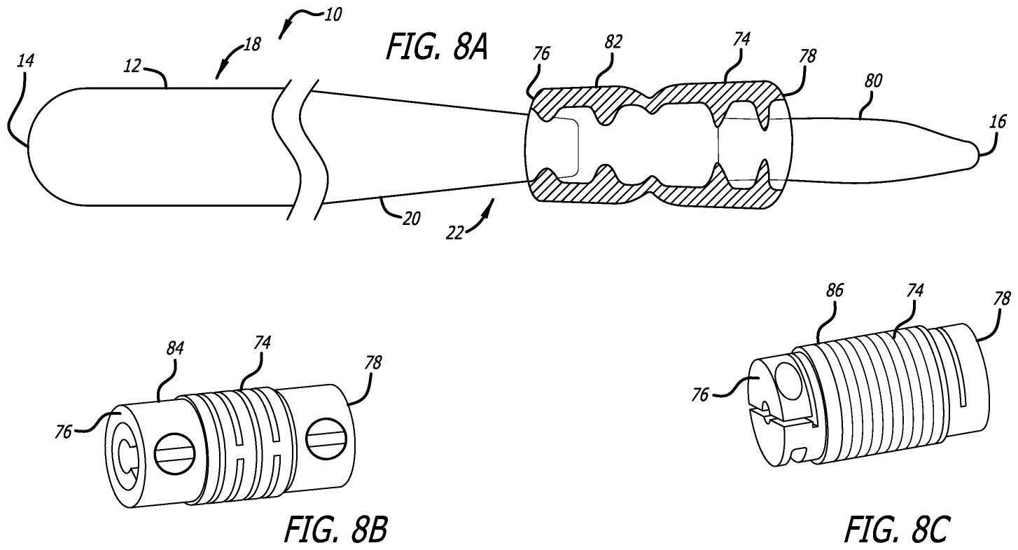

In another embodiment, shown in FIGS. 7A and 7B, a groove 70 is formed in the distal section 22 of guidewire 10 in order to form the weakened section 50. The groove 70 can be formed by grinding or by a laser and should have a smooth curved shape. The U-shaped section 30 is formed by bending the distal section 22 at the groove 70 to form the transition joint 28. The groove can be formed in either the round cross-section 26A of the constant diameter section 26 or the rectangular cross-section 27A of the constant rectangular section 27. The groove 70 can have an hourglass-shaped section, a U-shaped section, a V-shaped section, or be more curved than a U-shape, and still provide the weakened section 50 at the transition joint 38 so that the U-shaped section 30 can be formed.

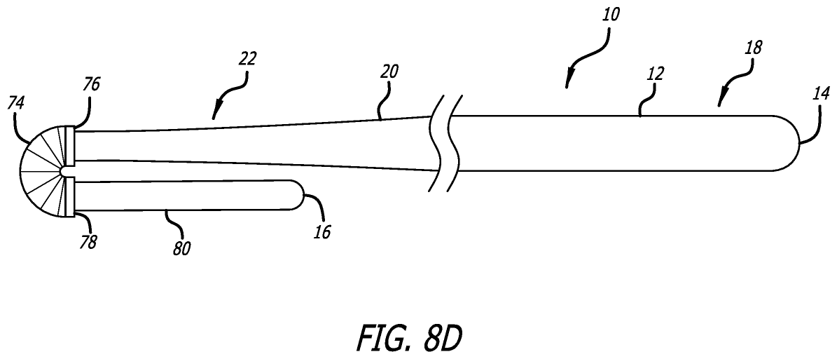

In another embodiment, shown in FIGS. 8A-8D, a dedicated flexible joint 74 is attached to the distal end 16 of the distal section 22. The flexible joint 74 has a proximal end 76 and a distal end 78 and is approximately 0.25 inch to 0.75 inch long. The flexible joint 74 is preferably hollow and highly flexible so that it can be bent into a U-shape. The distal end 16 of the distal section 22 is inserted into and attached to the proximal end 76 of the flexible joint 74. A distal wire 80 is inserted into and attached to the distal end 78 of the flexible joint 74. The flexible joint 74 can be formed of a nitinol connector 82 (FIG. 8A), a bellows connector 84 (FIG. 8B), or a coil wire connector 86 (FIG. 8C). For each of the nitinol connector 82, the bellows connector 84, or the coil wire connector 86, the design allows for the connectors to be bent up to 360° where one side of the connectors elongate and the opposite side compresses. As shown in FIG. 8D, one of the connectors 82, 84 or 86 can be used to form the weakened section 50 so that the connector 82, 84, or 86 can bend 180° to form the U-shaped section 30. The nitinol connector 82, bellows connector 84, and the coil wire connector 86 have enough flexibility to form the U-shaped section 30, and enough stiffness to hold the shape of the U-shaped section 30 when the guidewire 10 is advanced through a tortuous vessel or into a tight side branch vessel. In one embodiment, the bellows connector 84 is formed from a polymer material or a nitinol material.

For all of the embodiments disclosed herein, the outer curve of the U-shaped bend 46 has radius in a range from 0.01 inch to 0.17 inch.

While the invention has been illustrated and described herein in terms of its use as a guidewire, it will be apparent to those skilled in the art that the guidewire can be used in all vessels in the body. All dimensions disclosed herein are by way of example. Other modifications and improvements may be made without departing from the scope of the invention.

I claim:

1. A guidewire, comprising:
an elongated tubular member having a proximal end and a distal end;
a proximal section extending from the proximal end toward the distal end;
a distal section extending from the distal end toward the proximal end, the distal section having a tapered section and a constant diameter section distal of the tapered section;
a transition joint positioned between the tapered section and the distal end; and
an extended U-shaped section formed adjacent the transition joint.

2. The guidewire of claim 1, wherein the extended U-shaped section extends from the constant diameter section to the distal end with the transition joint positioned therebetween.

3. The guidewire of claim 2, wherein the extended U-shaped section has a first member having a first length, a second member having a second length, and a U-shaped bend joining the first member and second member.

4. The guidewire of claim 3, wherein the U-shaped bend has an outer curve having an arc length and a midpoint on the arc length.

5. The guidewire of claim 4, wherein the first length of the first member extends from the distal end to the midpoint on the arc length, and the second length of the second member is equal to the first length of the first member.

6. The guidewire of claim 5, wherein the second length of the second member extends from the midpoint of the arc length and along the constant diameter section of the distal section.

7. The guidewire of claim 6, wherein the first length of the first member is in a range from 0.07 inch to 2.5 inch.

8. The guidewire of claim 7, wherein the second length of the second member is in a range from 0.07 inch to 2.50 inch.

9. The guidewire of claim 8, wherein the extended U-shaped section has a length in a range from 0.07 inch to 2.50 inch.

10. The guidewire of claim 9, wherein the outer curve of the U-shaped bend has a radius in a range from 0.01 inch to 0.17 inch.

11. The guidewire of claim 10, wherein the constant diameter section has a weakened section on the second member and adjacent to the transition joint.

12. The guidewire of claim 11, wherein the weakened section is formed by heat treatment in a range from 350° C. to 600° C. for a time in a range from 30 seconds to 85 minutes on the second member adjacent to the transition joint.

13. The guidewire of claim 12, wherein the constant diameter section is formed from a superelastic material.

14. The guidewire of claim 13, wherein the superelastic material is Nitinol.

15. The guidewire of claim 11, wherein the weakened section is formed by any of a notch, an hourglass-shaped section, a groove, a U-shaped section, or a V-shaped section in the second member positioned adjacent to the transition joint.

16. The guidewire of claim 11, wherein the weakened section is formed by attaching a ribbon member to the second member and adjacent to the transition joint.

17. The guidewire of claim 11, wherein the weakened section is formed by positioning a flexible joint between the second member and the transition joint.

18. The guidewire of claim 11, wherein the weakened section is formed by positioning a bellows joint between the second member and the transition joint.

19. The guidewire of claim 10, wherein the constant diameter section has a strengthened section on the second member and adjacent the transition joint.

20. The guidewire of claim 19, wherein the strengthened section is formed by heat treatment in a range from 250° C. to 450° C. for a time in range from 2 minutes to 45 minutes on the second member at a point adjacent to the transition joint.

21. A guidewire, comprising:
an elongated tubular member having a proximal end and a distal end;
a proximal section extending from the proximal end toward the distal end;
a distal section extending from the distal end toward the proximal end, the distal section having a tapered section and a flattened wire section distal of the tapered section; and
a transition joint positioned between the tapered section and the distal end; and
an extended U-shaped section formed adjacent the transition joint.

22. The guidewire of claim 21, wherein the extended U-shaped section extends from the flattened wire section to the distal end with the transition joint positioned therebetween.

23. The guidewire of claim 22, wherein the extended U-shaped section has a first member having a first length, a second member having a second length, and a U-shaped bend joining the first member and second member.

24. The guidewire of claim 23, wherein the U-shaped bend has an outer curve having an arc length and a midpoint on the arc length.

25. The guidewire of claim 24, wherein the first length of the first member extends from the distal end to the midpoint on the arc length, and the second length of the second member is equal to the first length of the first member.

26. The guidewire of claim 25, wherein the second length of the second member extends from the midpoint of the arc length and along the flattened wire section of the distal section.

27. The guidewire of claim 26, wherein the first length of the first member is in a range from 0.07 inch to 2.50 inch.

28. The guidewire of claim 27, wherein the second length of the second member is in a range from 0.07 inch to 2.50 inch.

29. The guidewire of claim 28, wherein the extended U-shaped section has a length in a range from 0.07 inch to 2.50 inch.

30. The guidewire of claim 29, wherein the outer curve of the U-shaped bend has a radius in a range from 0.01 inch to 0.17 inch.

31. The guidewire of claim 30, wherein the flattened wire section has a weakened section on the second member and adjacent to the transition joint.

32. The guidewire of claim 31, wherein the weakened section is formed by heat treatment in a range from 350° C. to 600° C. for a time in a range from 3 minutes to 85 minutes on the second member adjacent to the transition joint.

33. The guidewire of claim 32, wherein the flattened wire section is formed from a superelastic material.

34. The guidewire of claim 33, wherein the superelastic material is Nitinol.

35. The guidewire of claim 31, wherein the weakened section is formed by any of a notch, an hourglass-shaped section, a groove, a U-shaped section, or a V-shaped section in the second member positioned adjacent to the transition joint.

36. The guidewire of claim 31, wherein the weakened section is formed by attaching a ribbon member to the second member and adjacent to the transition joint.

37. The guidewire of claim 30, wherein the flattened wire section has a strengthened section on the second member and adjacent the transition joint.

38. The guidewire of claim 37, wherein the strengthened section is formed by heat treatment in a range from 250° C. to 450° C. for a time in range from 2 minutes to 45 minutes on the second member at a point adjacent to the transition joint.

39. The guidewire of claim 12, wherein the heat treatment is applied to multiple sections of the constant diameter sections.

40. The guidewire of claim 20, wherein the heat treatment is applied to multiple sections of the constant diameter sections.

41. The guidewire of claim 32, wherein the heat treatment is applied to multiple sections of the constant diameter sections.

42. The guidewire of claim 38, wherein the heat treatment is applied to multiple sections of the constant diameter sections.
